(19) United States
(12) Patent Application Publication
(10) Pub. No.: US 2003/0163096 A1
(43) Pub. Date: Aug. 28, 2003

(54) Shieldable fluid collection set

(75) Inventors: Kirk D. Swenson, North Caldwell, NJ (US); C. Mark Newby, Tuxedo, NY (US); Jamieson William MacLean Crawford, New York, NY (US)

Correspondence Address: CASELLA & HESPOS LLP, 274 MADISON AVENUE, SUITE 1703, NEW YORK, NY, 10016-0701, US

(73) Assignee: Becton, Dickinson and Company, Franklin Lakes, NJ

(21) Appl. No.: 10/309,724

(22) Filed: Dec. 4, 2002

Related U.S. Application Data
(60) Provisional application No. 60/361,447, filed on Feb. 28, 2002.

(51) Int. Cl. A61M 5/00

(52) U.S. Cl. 604/263; 604/177

(57) ABSTRACT

A fluid collection or infusion set is provided with a needle assembly that is slidably disposed in a generally tubular shield. The needle assembly includes two opposed flexible wings that are slidably receivable in at least one slot of the shield. The wings can be rotated from a coplanar disposition into a position where the wings are in face-to-face engagement with one another. A flexible bridge may extend between the wings to facilitate manipulation of the needle assembly. The shield may be tapered to wider dimensions closer to the proximal end of the shield. The wings may include lugs that engage external surfaces of the shield and cause the wings to stretch as the shield is moved distally over the needle assembly. The lugs then snap into recesses at the proximal end of the shield or preventing re-exposure of the needle. Alternatively, the shield may include a locking clip that can be rotated into a position for covering portions of the slot in the shield to prevent re-exposure of the needle.

### RELATED APPLICATION

This application claims priority on U.S. Provisional Patent Appl. No. 60/361,447 which was filed on Feb. 28, 2002.

### FIELD OF THE INVENTION

The invention relates to a medical apparatus with a retractable needle for fluid collection or infusion.

### BACKGROUND OF THE INVENTION

Needle cannulas are employed for collecting blood or other bodily fluids from a patient or for infusing blood, drugs or other liquids into a patient. The needle cannula typically is mounted to a plastic hub, which in turn is mounted to a device for collecting or infusing the liquid. One such device includes a length of flexible plastic tubing with a distal end connected to the needle hub and a proximal end connected to a plastic fitting. The fitting at the proximal end of the plastic tubing can take many forms depending on the intended use of the device. Devices of this type often are referred to as blood collection sets, fluid collection sets or intravenous infusion sets, depending upon the intended use of the device.

The above-described medical devices often include a pair of flexible plastic wings mounted to or near the needle hub. The wings can be folded into face-to-face engagement with one another, and hence define a convenient handle for gripping and manipulating the needle cannula. The wings also can be rotated away from one another and can be taped into face-to-face contact with the skin of the patient.

Accidental sticks with a needle cannula can be painful and can transmit disease. As a result, most needle assemblies are employed with rigid means for enclosing the needle cannula both prior to use and after use. Protection prior to use typically is achieved by a rigid plastic tube that has a proximal end frictionally mounted to the needle hub and a distal end that extends beyond the distal end of the needle cannula. The plastic tube is removed and discarded immediately prior to use of the needle cannula. Protection after use of the needle cannula typically is achieved by a tubular shield that can be telescoped relative to the needle hub and needle cannula from a proximal position where the needle is enclosed to a distal position where the needle cannula is safely within the tubular shield. Shields of this type typically include means for releasably holding the shield in its proximal position and for holding the shield more securely in its distal position. The retention of the shield in its distal position should prevent any accidental re-exposure of the used needle cannula and preferably should prevent or substantially complicate an intentional attempt to reuse the needle cannula.

### SUMMARY OF THE INVENTION

The invention is directed to a fluid collection or infusion set that comprises a length of flexible plastic tubing with opposite proximal and distal ends and a passage extending between the ends. A fitting is securely connected to the proximal end of the flexible plastic tubing.

The fluid collection or infusion set further comprises a needle assembly. The needle assembly includes a needle hub with a proximal end, a distal end and a passage extending between the ends. Portions of the passage adjacent the proximal end of the hub are securely engaged with the distal end of the flexible plastic tubing.

The needle assembly further includes a needle cannula having opposite proximal and distal ends and a lumen extending between the ends. Thus, the lumen through the needle cannula communicates with the passage through the needle hub and with the passage through the flexible tubing. The needle assembly may further include a needle protector removably mounted over the needle cannula and extending sufficiently to cover the distal end of the needle cannula.

Two opposed flexible wings project transversely from the needle hub. The wings can be folded into substantially face-to-face relationship with one another to facilitate gripping of the needle assembly between a thumb and forefinger. Thus, the folded wings effectively function as a handle to facilitate manipulation of the needle assembly. The wings also can be folded into a substantially coplanar disposition for taping the needle assembly onto the skin of a patient. Additionally, the wings may include a flexible bridge extending from one wing to the other at locations spaced from the hinged connection of the wings to the needle hub. The bridge can be collapsed between the wings when the wings are folded into face-to-face engagement with one another. However, the bridge projects from the wings when the wings are in their coplanar disposition, and hence defines an actuator for facilitating movement of the needle assembly when the wings are in their coplanar disposition. Each of the wings may be formed with a lug projecting from the plane defined by each wing.

The fluid collection or infusion set further comprises a generally tubular shield telescoped over the needle assembly. The shield includes opposite proximal and distal ends and a passage extending between the ends. The needle hub is dimensioned to move slidably in the passage from a distal position where the needle cannula is exposed to a proximal position where the needle cannula is safely disposed within the shield. The shield includes two opposed slots that extend from a location at or near the distal end to a location at or near the proximal end. The slots are dimensioned to slidably accommodate the wings so that the needle assembly and the wings can be moved from the distal position to the proximal position relative to the shields. Proximal portions of the slots may be enlarged to define a recess into which the wings will be received when the needle assembly reaches the proximal position. Thus, the wings can be trapped in the recesses for complicating or preventing movement of the needle assembly back toward the distal position.

The external width of the shield may vary gradually from a minor dimension at the distal end of the slots to a major dimension at the proximal ends of the slots. More particularly, the external width of the shield at the distal end preferably is less than or equal to the distance between the lugs on the wings. However, the external width of the shield at the proximal end of the slots preferably exceeds the distance between the lugs. As a result, the initial proximal movement of the needle assembly relative to the shield can be carried out easily without interference between the lugs and the shield. Further movement, however, causes the lugs to engage the exterior of the shield. Hence, the wings stretch slightly away from one another as the needle assembly moves further in the proximal direction. The lugs align with the recesses at the proximal ends of the slots when the needle assembly reaches its proximal position. Thus, the wings return resiliently to an unstretched condition and the lugs move into the recess at the proximal end of the slot for securely trapping the needle assembly in the proximal position. A return distal movement of the needle assembly would require a very complicated stretching of the wings by hand while simultaneously displacing the wings and needle assembly distally relative to the shield.

In an alternate embodiment, the shield may comprise only a single slot and may have a cylindrical outer surface. The shield may further include a rotatable split clip mounted over the cylindrical outer surface of the shield. The clip may initially be in a rotational orientation with the split in the clip aligned with the slot in the shield. Shielding of this embodiment is achieved by rotating the wings into face-to-face engagement with one another and sliding the shield distally over the needle assembly so that the folded wings slide into and through the single slot of the shield and the slit in the rotatable clip. The clip may be rotated relative to the shield after the wings have moved proximally beyond the rotatable clip. Thus, the clip prevents a return distal movement of the needle assembly.

### BRIEF DESCRIPTION OF THE DRAWINGS

FIG. 1 is a perspective view of a fluid collection or infusion set in accordance with a first embodiment of the invention.

FIG. 2 is an end elevational view of the fluid collection or infusion set of FIG. 1.

FIG. 3 is an end elevational view similar to FIG. 2, but showing the wings in a folded condition.

FIG. 4 is a perspective view of a second embodiment of a fluid collection or infusion set in accordance with the subject invention with the needle assembly in a ready-to-use position.

FIG. 5 is a longitudinal cross-sectional view of the fluid collection set of FIG. 4 prior to shielding.

FIG. 6 is a cross-sectional view taken along line 6-6 in FIG. 5.

FIG. 7 is a cross-sectional view similar to FIG. 5, but showing the needle assembly in a partly shielded condition.

FIG. 8 is a cross-sectional view similar to FIGS. 5 and 7, but showing the needle assembly in a fully shielded condition.

FIG. 9 is a perspective view of a third embodiment of the fluid collection or infusion set in accordance with the subject invention with the needle assembly in a ready-to-use condition.

FIG. 10 is a perspective view of the fluid collection or infusion set of FIG. 9 with the needle assembly in a fully shielded condition.

### DETAILED DESCRIPTION OF THE INVENTION

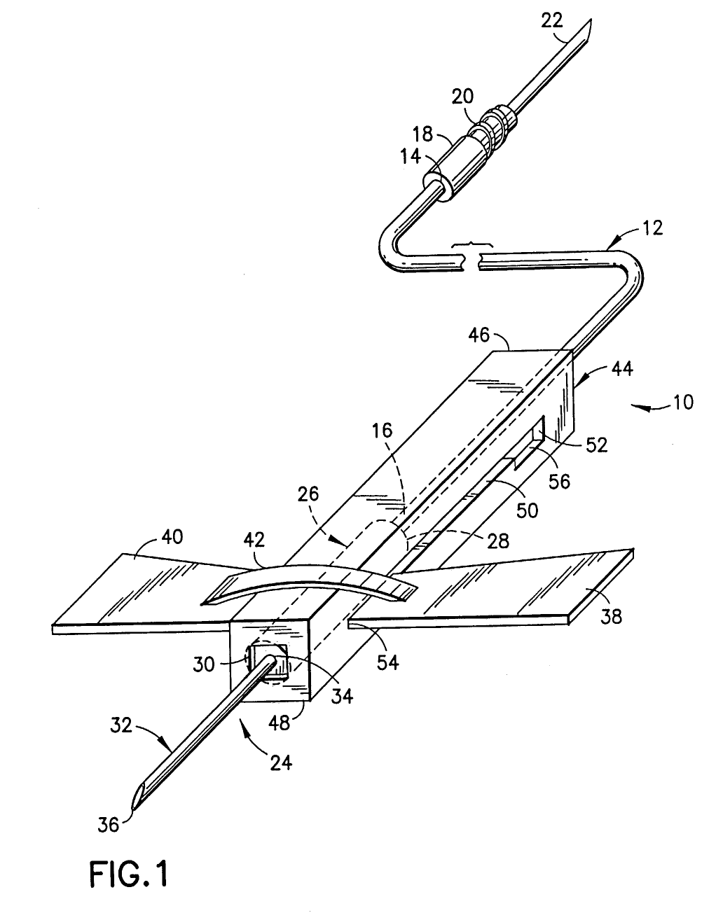

A fluid collection set in accordance with a first embodiment of the invention is identified generally by the numeral 10 in FIGS. 1-3. Fluid collection set 10 includes a length of flexible plastic tubing 12 with a proximal end 14 and a distal end 16. Proximal end 14 of flexible tubing 12 is connected to a plastic fitting 18. Fitting 18 can take many different forms. However, fitting 18 shown in FIG. 1 includes an array of external threads 20 and a proximal needle cannula 22 to enable fitting 18 to be connected with a holder (not shown) for receiving an evacuated blood collection tube.

Fluid collection set 10 further includes a needle assembly 24. Needle assembly 24 includes a plastic needle hub 26 with a proximal end 28, a distal end 30 and a passage (not shown) extending between the ends. Distal end 16 of plastic tubing 20 is connected to the passage of hub 26 adjacent proximal end 28 so that the passage through needle hub 26 communicates with the passage through flexible tubing 12. Needle assembly 24 further includes a needle cannula 32 with a proximal end 34, a distal end 36 and a lumen extending between the ends. Proximal end 34 of needle cannula 32 is securely mounted in the passage of needle hub 26 adjacent distal end 30 of needle hub 26.

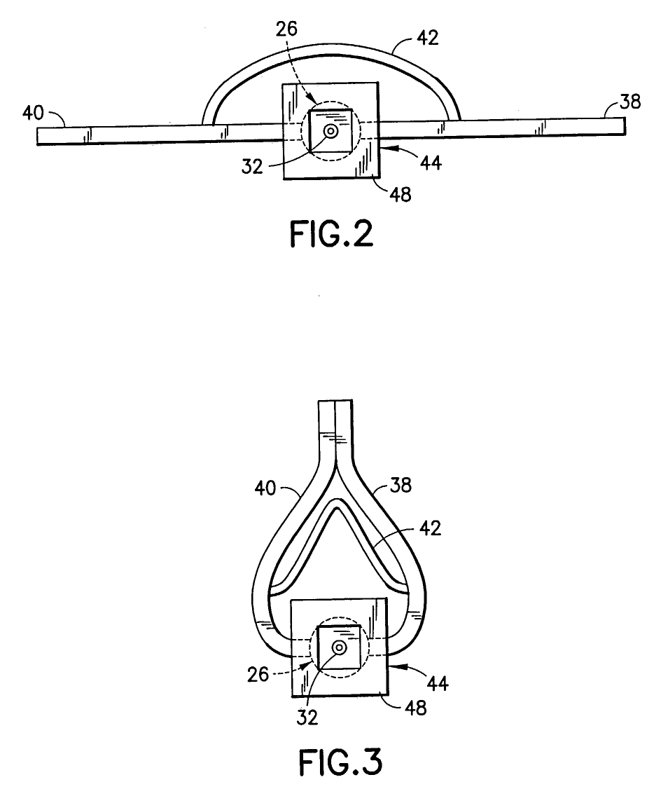

Needle assembly 24 further includes flexible wings 38 and 40 extending transversely from needle hub 26. Wings 38 and 40 can be rotated relative to needle hub 26 from the coplanar orientation shown in FIGS. 1 and 2 into the condition shown in FIG. 3 where at least upper portions of wings 38 and 40 are in substantially face-to-face engagement with one another.

Needle assembly 24 is further characterized by a collapsible bridge 42 extending between and connecting central positions on wings 38 and 40. Bridge 42 defines a generally arch shape when wings 38 and 40 are in the coplanar orientation shown in FIGS. 1 and 2. However, bridge 42 collapses upon itself when wings 38 and 40 are rotated toward one another and into the generally face-to-face disposition shown in FIG. 3.

Fluid collection or infusion set 10 further includes a generally tubular shield 44 with opposite proximal and distal ends 46 and 48 and a passage extending between the ends. The passage of tubular shield 44 is dimensioned to slidably accommodate needle hub 26 so that needle assembly 24 can be moved from a distal position shown in FIG. 1 where needle cannula 32 projects distally from shield 44 to a proximal position where needle cannula 32 is enclosed within shield 44.

Shield 44 is characterized by longitudinal slots 50. Each slot 50 includes a proximal end 52 near proximal end 46 of shield 44 and a distal end 54 near distal end 48 of shield 44. Portions of slots 50 extending proximally from distal end 54 are dimensioned to slidably engage portions of wings 38 and 40 inwardly from bridge 42. However, portions of slots 50 adjacent proximal end 52 define a recess 56 for locked engagement with wings 38 and 40.

Fluid collection or infusion set 10 initially is in the FIG. 1 orientation with needle cannula 32 projecting distally beyond shield 44. Wings 38 and 40 can be in the coplanar disposition shown in FIGS. 1 and 2, with bridge 42 extending between wings 38 and 40 and over portions of shield 44. Thus, bridge 42 defines an engagement surface that can facilitate one-handed manipulation of needle assembly 24. More particularly, proximal portions of shield 44 can be engaged between a thumb and forefinger, while an index finger may be disposed against bridge 42 to facilitate guiding and manipulation of needle assembly 24. Bridge 42 also is useful for moving needle assembly 24 from the distal position to the proximal position. In particular, proximal portions of shield 44 can be engaged between a thumb and forefinger, while the index finger of the same hand engages the distal side of bridge 42 and pulls needle assembly 24 into the proximal position.

Needle assembly 24 also can be manipulated by rotating wings 38 and 40 toward one another and into substantially face-to-face engagement as shown in FIG. 3. Thus, the user can grip outwardly facing surfaces of wings 38 and 40 between a thumb and forefinger for manipulating needle assembly 24.

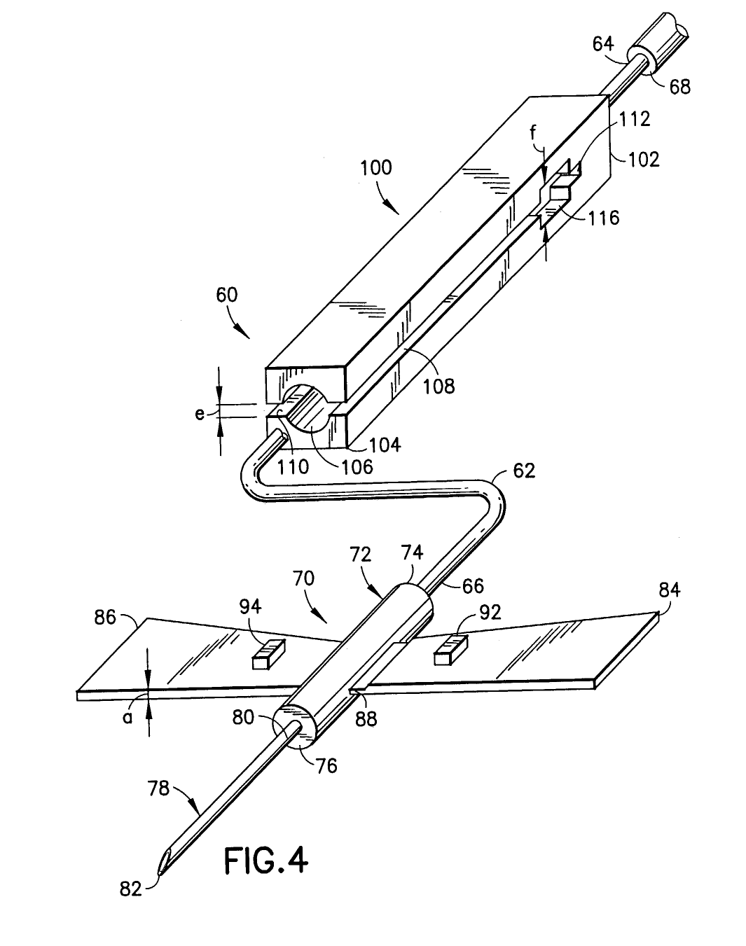

A second embodiment of the fluid collection or infusion set is identified by the numeral 60 in FIGS. 4-8. Fluid collection or infusion set 60 includes flexible tubing 62 with a proximal end 64 and a distal end 66. Proximal end 64 is connected to a fitting 68 that may be identical to fitting 18 shown in FIG. 1. Distal end 66 of flexible tubing 62 is connected to a needle assembly 70 that is similar to needle assembly 24 shown in FIGS. 1-3. More particularly, needle assembly 70 includes a needle hub 72 with a proximal end 74, a distal end 76 and a passage extending between the ends. Distal end 66 of plastic tubing 62 is connected securely to portions of the passage at proximal end 74 of needle hub 72. Thus, the passage through needle hub 72 communicates with the passage through flexible tubing 62. Needle assembly 70 further includes a needle cannula 78 having a proximal end 80 securely mounted in the passage of needle hub 72 adjacent distal end 76 of needle hub 72. Needle cannula 78 further includes a distal end 82 and a lumen extending between the ends.

Needle assembly 70 also includes flexible wings 84 and 86 extending transversely from needle hub 72. Wings 84 and 86 have a thickness “a” at most locations, but include thinned portions 88 and 90 adjacent needle hub 72. Thinned portions 88 and 90 facilitate articulation of wings 84 and 86 about needle hub 72. Wings 84 and 86 are formed from an elastomeric material and define a thickness at thin portions 88 and 90 that is selected to facilitate a slight resilient outward stretching of wings 84 and 86 away from needle hub 72.

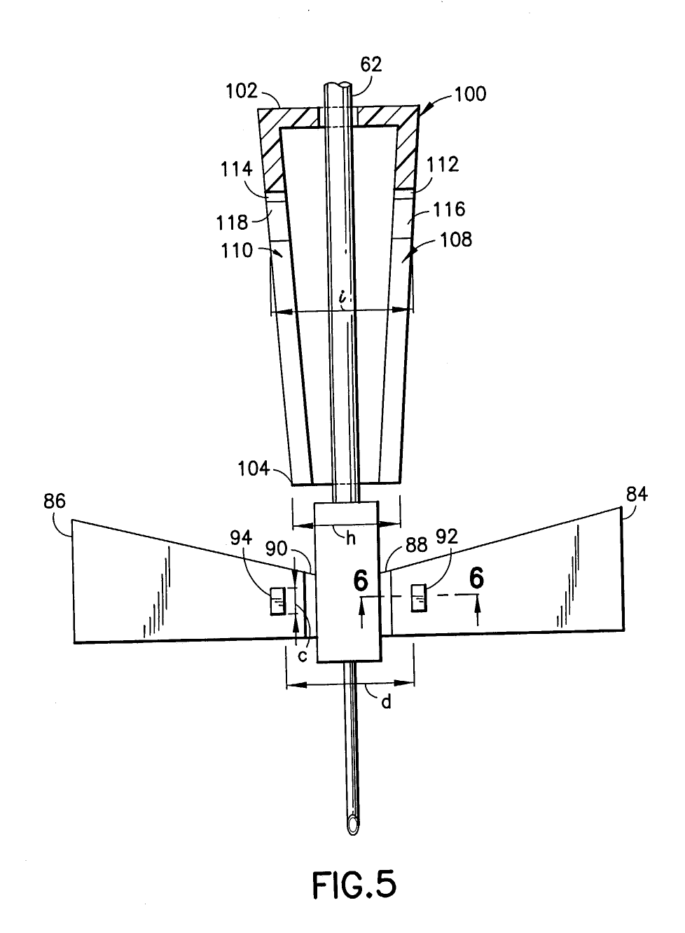

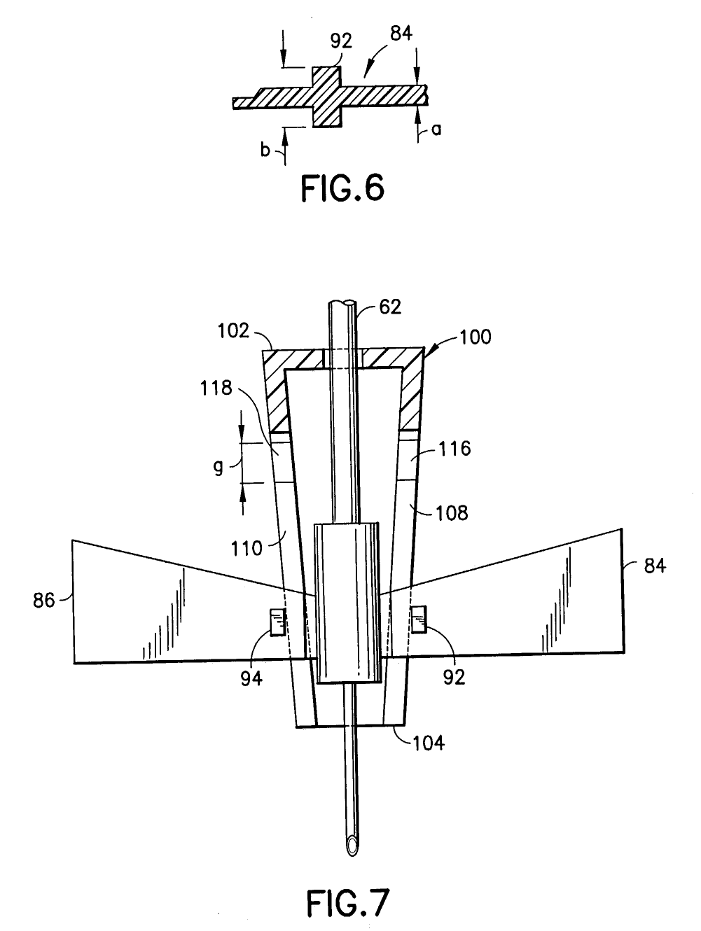

Wings 84 and 86 are formed with lugs 92 and 94 respectively projecting upwardly and downwardly from the top and bottom surfaces of wings 84 and 86 to define a thickness “b” as shown in FIG. 6 and a length “c” as shown in FIG. 5. Lugs 92 and 94 are spaced from one another by a distance “d” as shown in FIG. 5. Needle assembly 70 may further be provided with a bridge substantially identical to bridge 42 described and illustrated with respect to FIGS. 1-3. However, the bridge would have to be connected to wings 84 and 86 at locations outwardly from lugs 92 and 94. Bridge 42 was an important feature of the first embodiment depicted in FIGS. 1-3. However, a bridge is not required for the second embodiment.

Fluid collection or infusion set 60 further comprises a shield 100. Shield 100 is unitarily molded from a rigid plastic material and includes a proximal end 102, a distal end 104 and a passage 106 extending between the ends. Shield 100 is further characterized by slots 108 and 110 that extend proximally from distal end 104 of shield 100. Slots 108 and 110 are opposed to one another and substantially coplanar and extend entirely through peripheral walls of shield 100 from passage 106 to external locations. Additionally, slots 108 and 110 define a height “e” approximately equal to thickness “a” of wings 84 and 86 at locations near lugs 92 and 94. Slots 108 and 110 have proximal ends 112 and 114 and recesses 116 and 118 immediately distally of proximal end 112 and 114. Recesses define a height “f” that exceeds the height “e” of distal portions of slots 108 and 110. Additionally, heights “f” of recesses 116 and 118 are slightly greater than thicknesses “b” of wings 84 and 86 at lugs 92 and 94. Recesses 116 and 118 further define lengths “g” that are slightly greater than the lengths “c” of lugs 92 and 94.

Shield 100 defines an external width “h” at distal end 104 which is less than distance “d” between lugs 92 and 94. However, shield 100 flares to wider dimensions at locations closer to recesses 116 and 118. In particular, shield 100 defines an external width “i” adjacent recesses 116 and 118 that is greater than distance “c” between lugs 92 and 94.

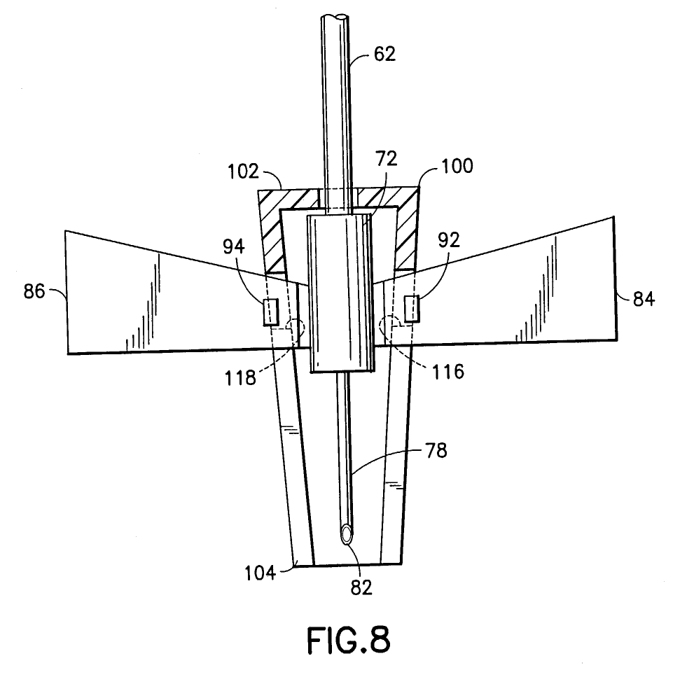

Fluid collection or infusion set 60 can be used substantially in a conventional manner with needle assembly 70 disposed distally of shield 100, as shown in FIG. 4. Thus, wings 70 can be rotated toward one another in a conventional manner for one-handed manipulation of needle assembly 70. After use, shield 100 is slid distally along tubing 62 and over proximal end 74 of needle hub 72. Thus, wings 84 and 86 will enter slots 108 and 110. Sufficient movement of shield 100 over needle assembly 70 will cause engagement between outer surfaces of shield 100 and lugs 92 and 94, as shown in FIG. 7. This engagement effectively creates ramping forces that will cause a stretching of thinned regions 88 and 90 of wings 84 and 86 to permit further movement of shield 100 over needle assembly 70. Sufficient distal advancement of shield 100 over needle assembly 70 will cause lugs 92 and 94 to align with recesses 116 and 118. As noted above, recesses 116 and 118 define height “f” and length “g” dimensions that exceed the corresponding dimensions “b” and “c” for lugs 92 and 94. Hence, wings 84 and 86 will resiliently return toward an unstretched condition and lugs 92 and 94 will enter recesses 116 and 118. In this position, as shown in FIG. 8, distal end 82 of needle cannula 78 is safely disposed in shield 100. The engagement of lugs 92 and 94 in recesses 116 and 118 will prevent a re-exposure of needle cannula 78. In particular, re-exposure would require an extremely complex stretching of wings 84 and 86 away from one another and simultaneous proximal movement of shield relative to needle assembly 70. However, flexible plastic tubing 62 is not structurally conducive to an accommodation of the forces that would be required to re-expose needle cannula 78.

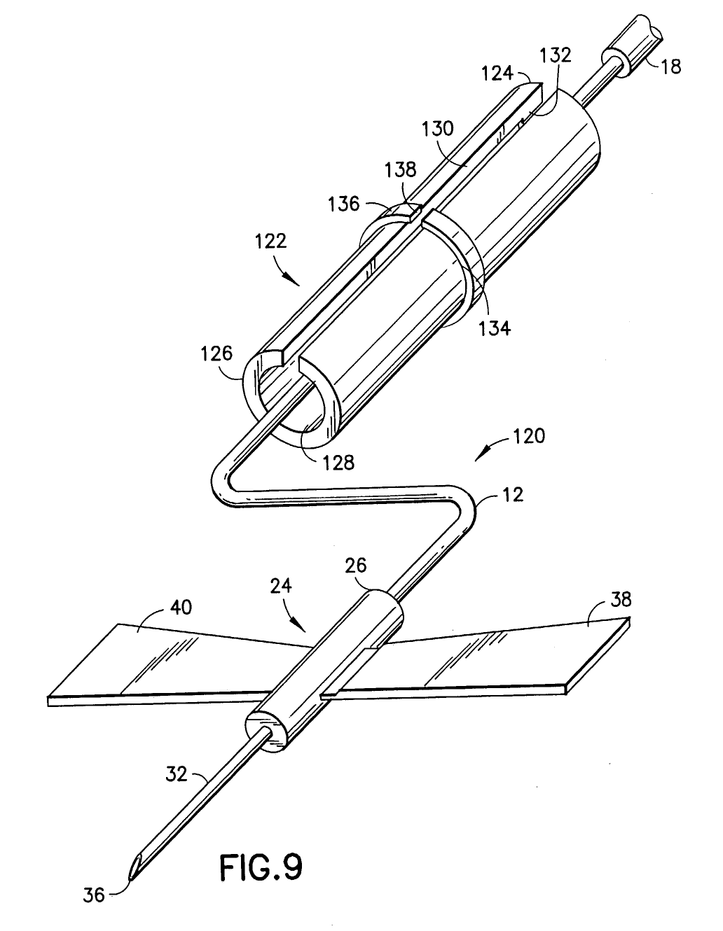

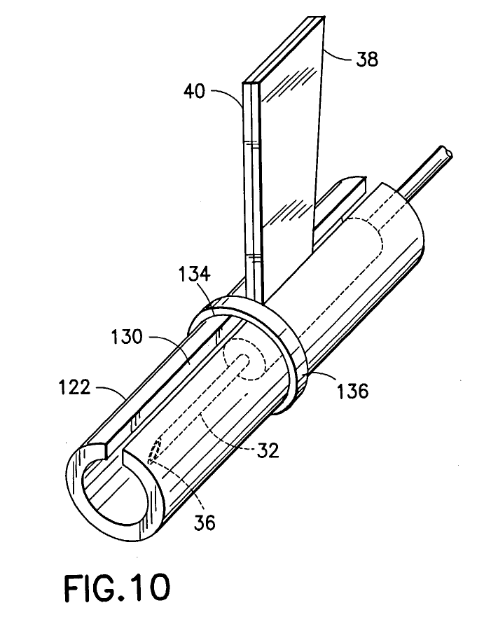

A third embodiment of the fluid collection or infusion set in accordance with the subject invention is identified generally by the numeral 120 in FIGS. 9-10. The fluid collection or infusion set 120 includes flexible plastic tubing 12, a fitting 18 and a needle assembly 24, all of which are substantially identical to comparably numbered elements in FIG. 1. However, fluid collection or infusion set 120 includes a shield 122 that is substantially different from the shield described and illustrated above. More particularly, shield 122 is a substantially cylindrical tube with a proximal end 124, a distal end 126 and a passage 128 extending between the ends. Passage 128 is dimensioned to slidably accommodate hub 26 of needle assembly 24. Shield 122 further includes a longitudinal slot 130 that extends from distal end 126 toward proximal end 124. Slot 130 is dimensioned to accommodate sliding movement of wings 38 and 40 when the wings are folded into face-to-face engagement with one another. Thus, needle assembly 24 can be slid in a distal-to-proximal direction within shield 122 with folded wings 38 and 40 slidably moving through slot 130. In the illustrated embodiment, slot 130 extends entirely to proximal end 124 of shield 122. Proximal end 124 is characterized further by an inwardly extending flange 132 that limits proximal movement of needle assembly 24 in shield 122. In other embodiments, however, slot 130 may terminate short of proximal end 124, and hence the proximal end of slot 130 will limit proximal movement of needle assembly 24 relative to shield 122.

Shield 122 further includes an annular groove 134 at an outer circumferential position thereon. A split annular clip 136 is rotatably mounted in groove 134. Clip 136 includes a slit 138 defining a width approximately equal to the width of slot 130. Clip 136 initially is in a rotatable position on shield 122 such that slit 138 of clip 136 aligns with slot 130 in shield 122. However, clip 136 can be rotated into a position where slit 138 and longitudinal slit 130 are misaligned.

Fluid collection set 120 is used substantially in a conventional manner with needle assembly 24 disposed distally of shield 22, as shown in FIG. 9. After use, wings 38 and 40 of needle assembly 24 are folded into face-to-face relationship with one another and shield 122 is slid distally relative to needle assembly 24. Folded wings 38 and 40 move longitudinally through slot 130 of shield 122. When needle assembly 24 reaches the extreme proximal position in shield 122, distal end 36 of needle cannula 32 is safely disposed within shield 122. At this time, wings 38 and 40 are disposed proximally of clip 136. Clip 136 then is rotated in annular groove 134 of shield 122 from the FIG. 9 position, where slit 138 aligns with slot 130 to the position shown in FIG. 10 where clip 136 prevents a return distal movement of needle assembly 24 relative to shield 122. Clip 136 and groove 134 may be formed with locking structures to prevent or substantially complicate a return rotational movement that could permit a re-exposure of needle cannula 32. For example, groove 134 may be formed with a locking recess, and shield 136 may be formed with a locking projection that engages in the locking recess.

What is claimed is:

1. A fluid collection set comprising a needle assembly having a needle hub, a needle cannula projecting from said hub, first and second flexible wings projecting transversely from said hub and being foldable into substantially face-to-face relationship with one another, a bridge connecting said first wing to said second wing at locations spaced from said needle hub, such that said bridge defines a finger actuation region for facilitating manipulation of said needle assembly, a shield slidably movable relative to said needle assembly from a proximal position where said needle cannula is exposed to a distal position where said needle cannula is shielded, said shield including at least one longitudinal slot for slidably receiving said wings, said shield further comprising at least one lock for locking said needle assembly in said proximal position relative to said shield.

2. The fluid collection set of claim 1, wherein said shield includes opposite proximal and distal ends, said at least one slot comprising first and second slots dimensioned for slidably receiving said first and second wings respectively, such that said needle hub is slidably disposed in said shield and such that said wings project respectively through said first and second slots, said bridge being disposed externally of said shield.

3. The fluid collection set of claim 2, further comprising a length of flexible tubing connected to said needle hub and a fitting connected to said end of said flexible tubing remote from said needle hub.

4. The fluid collection set of claim 3, wherein said fitting comprises an array of external threads, a needle cannula projecting from said fitting.

5. The fluid collection set of claim 2, wherein at least portions of said wings adjacent said needle hub are resiliently stretchable, said wings each being formed with a lug projecting therefrom, said shield being tapered from a minor width adjacent said distal end to a major width further from said distal end, said major width being greater than said distance between said lugs on said wings, said slots including recesses at proximal ends of said slots, said recesses being dimensioned for receiving said lugs, whereby said wings stretch as said major width of said shield approaches said lugs, and whereby said wings resiliently return to an unstretched condition with said lugs in said recesses when said recesses and said lugs align.

6. A fluid collection set comprising a needle assembly having a needle hub, a needle cannula projecting from said hub, first and second wings projecting transversely from said hub, said wings each being formed with a lug projecting therefrom, at least portions of said wings adjacent said hub being resiliently stretchable, said lugs being spaced from one another by a selected distance, said fluid collection set further comprising a shield having a proximal end, a distal end and a passage extending between said ends, said needle assembly being slidably movable in said passage from a distal position where said needle cannula is exposed to a proximal position where said needle cannula is within said shield, said shield including first and second slots slidably receiving said first and second wings respectively, each said slot having a proximal end defining a recess dimensioned for accommodating said lugs, portions of said slots distally of said recesses being cross-sectionally smaller than said lugs, portions of said shield adjacent said distal end being narrower than said distance between said lugs, said shield being tapered such that portions of said shield adjacent said recess are wider than said distance between said lugs, whereby movement of said needle assembly from said distal position to said proximal position causes said lugs to engage said shield for stretching said wings, said wings resiliently returning to an unstretched condition when said needle assembly is in said proximal position, such that said lugs engage in said recesses for locking said needle assembly in said proximal position.

7. The fluid collection set of claim 6, further comprising a length of flexible tubing connected to and extending from said needle hub, a fitting connected to an end of said flexible tubing remote from said needle hub.

8. The fluid collection set of claim 7, further comprising a proximal needle cannula mounted to said fitting, said fitting further comprising an array of threads for releasable connection with an evacuated tube holder.

9. The fluid collection set of claim 6, further comprising a bridge extending between said first and second wings, said lugs being disposed between connections of said bridge to said wings.

10. A fluid collection set comprising a needle assembly having a needle hub, a needle cannula projecting from said hub, first and second flexible wings projecting transversely from said hub and being foldable into substantially face-to-face relationship with one another, a shield slidably movable relative to said needle assembly from a proximal position where said needle cannula is exposed to a distal position where said needle cannula is shielded, said shield including a longitudinal slot dimensioned for receiving said wings when said wings are folded into said face-to-face relationship with one another, a split clip ring having a slit formed therein, said split clip ring being mounted on said shield and being rotatable from a first position where said slit in said split clip ring aligns with said slot to a second position where said slit is offset from said slot, said split clip ring being disposed at a longitudinal position along said shield to be distally of said wings when said shield is in said distal position on said needle assembly, whereby rotation of said split clip ring when said shield is in said distal position on said needle assembly prevents re-exposure of said needle cannula.

11. The fluid collection set of claim 9, further comprising locking means for securing said split clip ring in said position where said slit in said split clip ring is offset rotationally from said slot in said shield.

12. The fluid collection set of claim 10, wherein said shield includes means for preventing said shield from moving distally beyond said needle assembly.

13. The fluid collection set of claim 12, wherein the means comprises a flange extending inwardly at said proximal end of said shield.

14. The fluid collection set of claim 10, wherein said shield includes an annular groove, said split clip ring being rotatably engaged in said annular groove.
